(19) United States
(12) Patent Application Publication
(10) Pub. No.: US 2025/0366792 A1
(43) Pub. Date: Dec. 4, 2025

(54) SLEEP EVALUATION DEVICE, PROGRAM, SLEEP EVALUATION METHOD, AND LEARNING DEVICE

(71) Applicant: TOHOKU UNIVERSITY, Sendai-shi (JP)

(72) Inventors: Mitsuyuki NAKAO, Sendai-shi (JP); Yoichi TAKAHASHI, Sendai-shi (JP); Kentaro INUI, Sendai-shi (JP)

(21) Appl. No.: 19/216,154

(22) Filed: May 22, 2025

(30) Foreign Application Priority Data
May 31, 2024 (JP) 2024-089571

(51) Int. Cl. A61B 5/00

(52) U.S. Cl. CPC A61B 5/7267; A61B 5/4806; A61B 5/742

(57) ABSTRACT

A sleep evaluation device includes: an input unit configured to receive operation history information that is information indicating an operation history and a circadian rhythm pattern labeled to the operation history information from an external device; a machine learning unit configured to perform supervised learning for a circadian rhythm classification, thereby a model is obtained to classify a circadian rhythm based on training data including the operation history information of a first user and the circadian rhythm pattern labeled to the operation history information received; a classification unit configured to classify a circadian rhythm of a second user based on the operation history information of the second user received and the circadian rhythm classification model; and a reporting unit configured to report to the second user, the circadian rhythm classified by the classification unit.

### CROSS-REFERENCE TO RELATED APPLICATIONS

The present application is based upon and claims the right of priority to JP Patent Application No. No. 2024-089571, filed May 31, 2024, the disclosure of which is hereby incorporated by reference herein in its entirety for all purposes.

### TECHNICAL FIELD

The present invention relates to a sleep evaluation device, a program, a sleep evaluation method, and a learning device.

### BACKGROUND ART

Some people suffer from sleep disorders. According to the International Classification of Sleep Disorders (ICSD-3), sleep disorders are classified into seven groups of insomnia, sleep-related breathing disorders, central hypersomnia, circadian sleep-wake rhythm disorders, parasomnias, sleep-related movement disorders, and other sleep disorders. The circadian sleep-wake rhythm disorders, as one type of the sleep disorders, are classified into six groups: delayed sleep-wake phase disorder, advanced sleep-wake phase disorder, irregular sleep-wake rhythm disorder, non-24-hour sleep-wake rhythm disorder, shift work disorder, and jet lag. In a case of the circadian sleep-wake rhythm disorders, it may take 10 years or more for one to receive an accurate diagnosis after first asking for help from a medical institution for sleep disorder, or one may receive misdiagnoses multiple times over many years (see Non-Patent Document 1). One of criteria for diagnosing the circadian sleep-wake rhythm disorders is sleep diary or actigraph measurement. For example, Patent Document 1 below discloses a method of estimating a circadian rhythm of a living body based on a temporal change in length of body hair in a growth period obtained by identifying the body hair in the growth period in each of a plurality of images.

### CITATION LIST

### Patent Document

Patent Document 1: JP 2016-52414 A

### Non-Patent Document

[Non-Patent Document 1] Peter Mansbach, James S. P. Fadden, Lynn McGovern. Registry and survey of circadian rhythm sleep-wake disorder patients. Sleep Med X. December 2023.
[Non-Patent Document 2] Mitsuyuki Nakao, Yoichi A Taka Hashi, Akihiro Karashima, Ikuko Motoike, Tomohiro Katayama, Kengo Kinoshita, Kentaro Inui. Large-scale global analysis of rest-activity rhythm based on tweet frequency sequence. The Japanese Society of Sleep Research 45th Annual Meeting/30th Annual Meeting of the Japanese Society for Chronobiology. September 2023.

### SUMMARY OF INVENTION

### Technical Problem

However, sleep diaries and actigraph measurements need to be recorded for a long period of time ranging from several weeks to several months to be usable as the criteria for the diagnostic, depending on the type of circadian sleep-wake rhythm disorders and individual differences. The recording of sleep diaries is cumbersome and requires a great deal of effort when it goes on for a long period of time. Actigraph measurements are currently off-label and costly, and the typical duration of use is about two weeks. For example, according to the technique disclosed in Patent Document 1, it is necessary to acquire images of the skin a plurality of times, meaning that implementation thereof for a long period of time is unrealistic. For this reason, there is a need for a measurement device used instead of a sleep diary, actigraph measurement, or the like without placing a burden on the user.

In addition, the circadian sleep-wake rhythm disorders are not so well recognized, and many people may have the symptom without knowing the name of the disorders. For example, Patent Document 2, under a limitation of X (formerly Twitter (registered trademark)), suggests that about 1% of about 6,850,000 user IDs may have the non-24-hour sleep-wake rhythm disorder (non24 for short) which is one type of the circadian sleep-wake rhythm disorders. However, very few people are expected to be aware of the fact that he or she has the non-24-hour sleep-wake rhythm disorder (non24 for short). A task to be overcome by the present invention relates to a solution, employed instead of a sleep diary and actigraph measurement, for classifying a circadian rhythm from a long-term history of daily operations on a home appliance, an application, and the like and determining a symptom suspected to be the circadian sleep-wake rhythm disorders.

In view of the above circumstances, it is an object of the present invention to provide a technique for providing an opportunity to visit a medical institution at an early stage by indicating a symptom suspected to be the circadian sleep-wake rhythm disorder.

### Solution to Problem

One aspect of the present invention related to a sleep evaluation device including: an input unit configured to receive training data including operation history information and a circadian rhythm pattern labeled to the operation history information from an external device; a machine learning unit configured to perform supervised learning for a circadian rhythm classification thereby a model is obtained to classify a circadian rhythm based on training data including the operation history information of a first user and the circadian rhythm pattern labeled to the first user's operation history information received; a classification unit configured to classify a circadian rhythm of a second user's operation history information received based on the circadian rhythm classification model obtained; and a reporting unit configured to report to the second user, the circadian rhythm classified by the classification unit.

One aspect of the present invention is a program for causing a computer to function as the sleep evaluation device.

One aspect of the present invention is a sleep evaluation method including: receiving training data including operation history information that is time-series data indicating an operation history and a circadian rhythm pattern labeled to the operation history information from an external device; performing supervised learning for a circadian rhythm classification thereby a model is obtained to classify a circadian rhythm based on training data including the operation history information of a first user and the circadian rhythm pattern labeled to the first user's operation history information received; classifying a circadian rhythm of a second user's operation history information received based on the circadian rhythm classification model obtained; and reporting to the second user, the circadian rhythm classified through the classifying.

One aspect of the present invention is a learning device including a controller configured to perform supervised learning for a circadian rhythm classification thereby a model is obtained to classify a circadian rhythm based on training data including operation history information that is time-series data indicating an operation history received from an external device and a circadian rhythm pattern labeled to the operation history information.

### Advantageous Effects of Invention

According to the present invention, a symptom suspected to be a circadian sleep-wake rhythm disorder can be pointed out from a history of daily operations on a home appliance, an application, or the like, thereby making it possible to provide an opportunity to visit a medical institution at an early stage.

### BRIEF DESCRIPTION OF DRAWINGS

FIG. 1 is a conceptual diagram of the present invention.

FIG. 2 is an explanatory diagram illustrating a sleep evaluation device according to a first embodiment.

FIG. 3 is a diagram illustrating a first example of an image satisfying an image condition according to the first embodiment.

FIG. 4 is a diagram illustrating a second example of an image satisfying an image condition according to the first embodiment.

FIG. 5 is a diagram illustrating a third example of an image satisfying the image condition according to the first embodiment.

FIG. 6 is a diagram illustrating a fourth example of an image satisfying the image condition according to the first embodiment.

FIG. 7 is a diagram illustrating a fifth example of an image satisfying the image condition according to the first embodiment.

FIG. 8 is a diagram illustrating a sixth example of an image satisfying the image condition according to the first embodiment.

FIG. 9 is a diagram illustrating an example of a hardware configuration of the sleep evaluation device according to the first embodiment.

FIG. 10 is a diagram illustrating an example of a configuration of a controller of the sleep evaluation device according to the first embodiment.

FIG. 11 is a flowchart illustrating an example of a flow of processing executed by the sleep evaluation device according to the first embodiment.

FIG. 12 is a diagram illustrating an example of a hardware configuration of an information processing device (an example of a learning device) according to a second embodiment.

FIG. 13 is a flowchart illustrating an example of a flow of processing executed by the information processing device according to the second embodiment.

### DESCRIPTION OF EMBODIMENTS

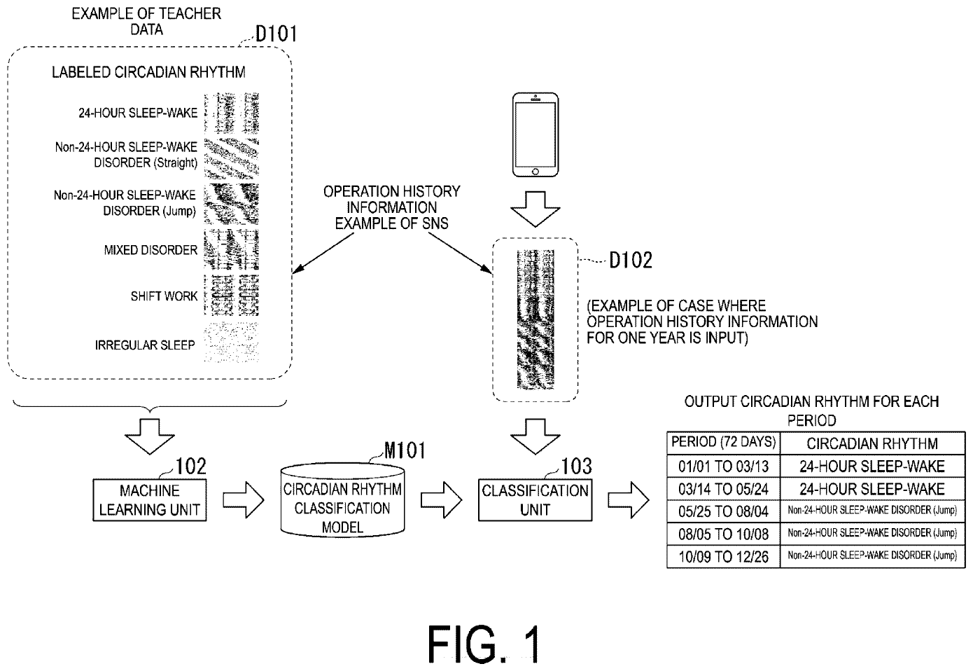

FIG. 1 is a conceptual diagram of the present invention. In FIG. 1, an input unit and a reporting unit are not illustrated. A machine learning unit 102 is a functional unit which will be described in detail below. Thus, in the example in FIG. 1, the machine learning unit 102 performs supervised learning for a circadian rhythm classification thereby a model is obtained to classify a circadian rhythm based on training data including operation history information of a first user input, and a circadian rhythm pattern labeled to the operation history information. In the example in FIG. 1, data D101 is an example of the training data including the operation history information of the first user and the circadian rhythm pattern labeled to the operation history information.

A classification unit 103 is a functional unit which will be described in detail below. Therefore, in the example in FIG. 1, the classification unit 103 classifies the circadian rhythm of a second user based on the operation history information of the second user input based on the circadian rhythm classification model. In the example in FIG. 1, information D102 is an example of the operation history information of the second user. In the example in FIG. 1, the model M101 is an example of a circadian rhythm classification model.

As the training data, for example, data that reliably represents the sleep-wake rhythm may be used. The circadian rhythm pattern is manually labeled to the operation history information. The labeling may be based on other types of information such as the presence or absence of a word such as “night shift” in the posted content in a case of X (formerly Twitter) for example. A person who labels the circadian rhythm pattern preferably has expert knowledge about the circadian rhythm.

### First Embodiment

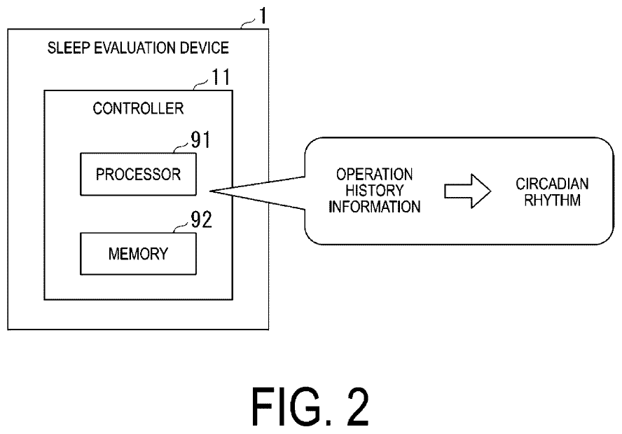

FIG. 2 is an explanatory diagram illustrating a sleep evaluation device 1 according to a first embodiment. The sleep evaluation device 1 includes a controller 11 including a processor 91 such as a central processing unit (CPU), a graphics processing unit (GPU), or a neural network processing unit (NPU) and a memory 92, which are connected to each other via a bus.

The controller 11 executes input acquisition processing, machine learning processing, classification processing, and report control processing. The input acquisition processing is processing for receiving information indicating an operation history from an external device. The external device is another device different from the sleep evaluation device. In other words, the input acquisition processing is a processing in which the controller 11 acquires information indicating an operation history input to the sleep evaluation device from an external device which is another apparatus different from the sleep evaluation device.

Note that the operation history is a history of date and time information indicating when a predetermined operation target is operated. The operation is, for example, turning ON/OFF the power supply to the operation target. The operation is, for example, an input using a keyboard. The operation is, for example, data transmission from an application. Note that what is required is the operation date and time information only, and the content of the operation (ON/OFF for example) is not the question. The operation target is, for example, a predetermined terminal such as a smartphone, a personal computer, or smart glasses.

The operation target may be, for example, a home appliance such as a refrigerator, a microwave oven, an air conditioner, a TV, a speaker, or an electric fan. The operation target may be, for example, a remote controller. The operation target may be, for example, a switch that switches ON and OFF the electricity in a room. The operation target may be, for example, an application that can be used in a terminal such as a smartphone, a personal computer, or smart glasses. Such an application may be, for example, an interactive application (or a messenger application) or may be, for example, an electronic bulletin board. The operation history is used instead of the sleep diary and the actigraph measurement.

The machine learning processing is processing of performing supervised learning for a circadian rhythm classification using training data including operation history information and a circadian rhythm pattern labeled to the operation history information. The circadian rhythm classification is performed based on a model obtained through the supervised learning of training data including input operation history information of the circadian rhythm classification target that is the first user input and the circadian rhythm pattern labeled to the operation history information. When the operation history information is data indicating the date and time, for example, a point indicating the date and time of the operation may be plotted on a white background image formed of pixels in a predetermined range (96×72), and an image as a result of the plotting may be used as the operation history information.

In the learning, the circadian rhythm classification model is created by repeatedly using a large amount (preferably about 100,000 pieces or more in order to realize high classification accuracy) of training data including the operation history information of the circadian rhythm classification target that is the first user and a circadian rhythm pattern labeled to the operation history information.

As will be described below, the circadian rhythm includes classifications such as 24-hour sleep-wake, non-24-hour sleep-wake disorder (Straight), non-24-hour sleep-wake disorder (Jump), mixed disorder (24-hour sleep-wake+non-24-hour sleep-wake rhythm disorders), shift work, and irregular sleep.

The classification processing is processing of classifying the circadian rhythm of the classification target based on the operation history information of the circadian rhythm classification target and the circadian rhythm classification model. Therefore, the classification processing is, for example, processing of classifying the circadian rhythm of the second user based on the operation history information of the second user input and the circadian rhythm classification model.

Note that the operation history information of an n-th user (n is 1 or 2) is information indicating a history of the n-th user's operation on the predetermined operation target. The operation target (for example, a smartphone) of the operation history information of the first user used in the machine learning processing may be the same as or different from the operation target (for example, a messenger application) of the operation history information of the second user subjected to the classification processing.

The report control processing is processing of controlling an operation of a predetermined reporting device (hereinafter referred to as a “reporter”) capable of issuing a report and reporting the circadian rhythm classified by the classification processing to the classification target of the classification processing. Therefore, the report control processing is processing of controlling the operation of the reporter to report the circadian rhythm classified by the classification processing to the second user. The reporter may be provided in an interface unit 12 described below for example.

### <Example of Effect of Controller 11>

The controller 11 executes classification processing. For the classification processing, a circadian rhythm classification model is used. The circadian rhythm classification model is a model for classifying the circadian rhythm of a user into types. When a disorder is found and the user can recognize the type of the disorder, he or she can be aware of the cause of his or her complaint. Therefore, the sleep evaluation device can provide an opportunity for the user to visit a medical institution at an early stage by pointing out a symptom suspected to be a circadian sleep-wake rhythm disorder.

The circadian rhythm classification model classifies a circadian rhythm based on the operation history information. Since the operation history information is information indicating an operation history and the operation history is a history of operations, it can be regarded that the circadian rhythm classification model classifies a circadian rhythm based on the operation history. The operation target may be operated under sleep blur. Still, that is an extremely rare case. Thus, an operation on the operation target indicates that the user (circadian rhythm classification target) is awake. Thus, the operation history information can be regarded as information indicating the timing at which the user is awake. The operation history information can also be regarded as information indicating a possibility of a timing at which the user is sleeping. Thus, the operation history information can be regarded as sleep-related information.

In the classification processing executed, a trained circadian rhythm classification model obtained by the machine learning processing executed in the preceding stage is used for the classification.

### <Operation History Information>

The operation history information will be further described. The operation history information may be indicated by, for example, time-series data indicating an operation history (hereinafter referred to as “operation history data”). The operation history information may be represented by an image of an operation history (hereinafter referred to as a “image data”). The image data may be generated based on the operation history data, for example.

The image data may be, for example, an image satisfying an image representation condition. The image representation condition is the following condition. Specifically, one of a vertical axis and a horizontal axis in a coordinate indicates a date and the other indicates time, and the range of time axis is n times longer than 24 hours, n being an integer that is one or more.

### <Example of Image Data Satisfying Image Representation Conditions>

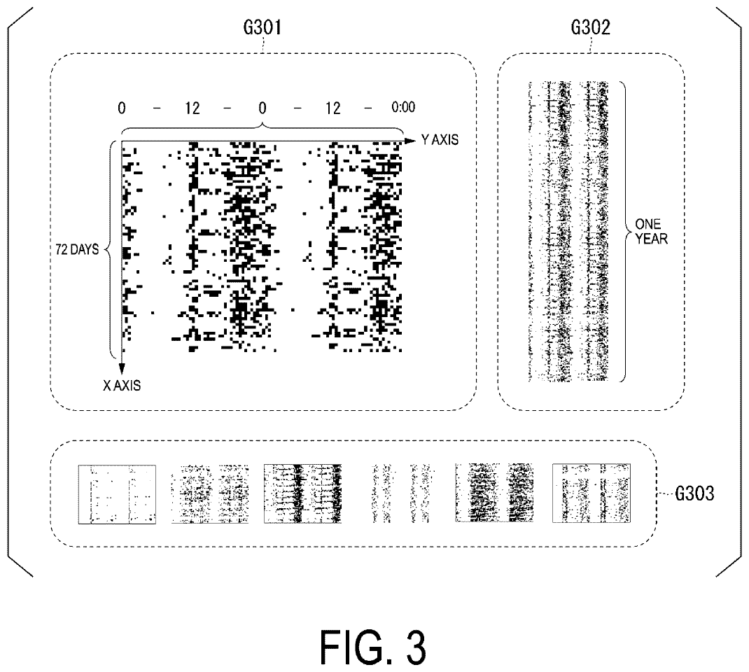

Examples of image data satisfying the image representation conditions will be described with reference to FIG. 3 to FIG. 8.

FIG. 3 to FIG. 8 are diagrams illustrating examples of a circadian rhythm represented by an image satisfying the image representation conditions according to the first embodiment.

FIG. 3 illustrates an image G301, an image G302, and an image G303. The image G301 is the operation history information of the first user, illustrating an example where the circadian rhythm indicates 24-hour sleep-wake rhythm. The image G303 is another example of the image G301. The image G302 illustrates an example of operation history information for one year for checking a difference from other circadian rhythms.

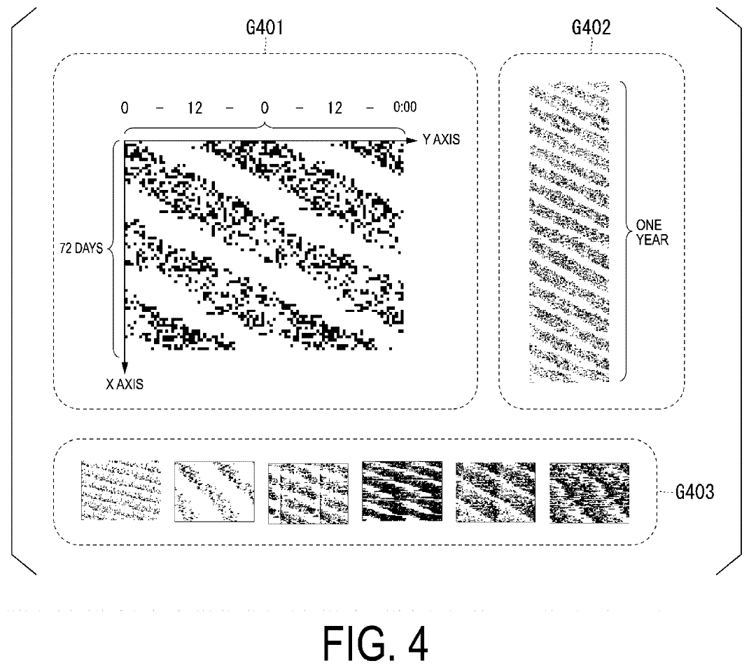

FIG. 4 illustrates an image G401, an image G402, and an image G403. The image G401 is the operation history information of the first user illustrating an example where the circadian rhythm indicates non-24-hour sleep-wake rhythm disorder (Straight). The image G403 is another example of the image G401. The image G402 illustrates an example of operation history information for one year for checking a difference from other non-24-hour rhythms (Straight).

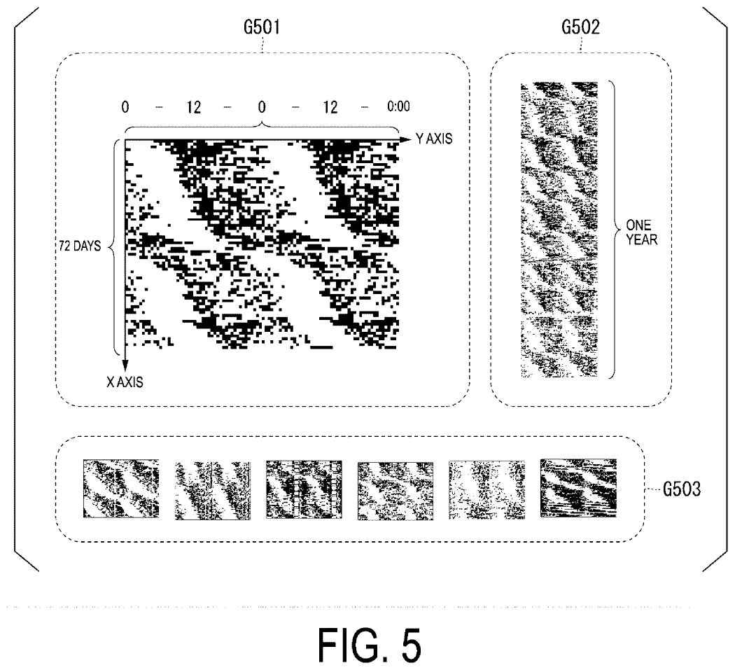

FIG. 5 illustrates an image G501, an image G502, and an image G503. The image G501 is the operation history information of the first user illustrating an example where the circadian rhythm indicates non-24-hour sleep-wake rhythm disorder (Jump). The image G503 is another example of the image G501. The image G502 illustrates an example of operation history information for one year for checking a difference from other non-24-hour rhythms (Jump).

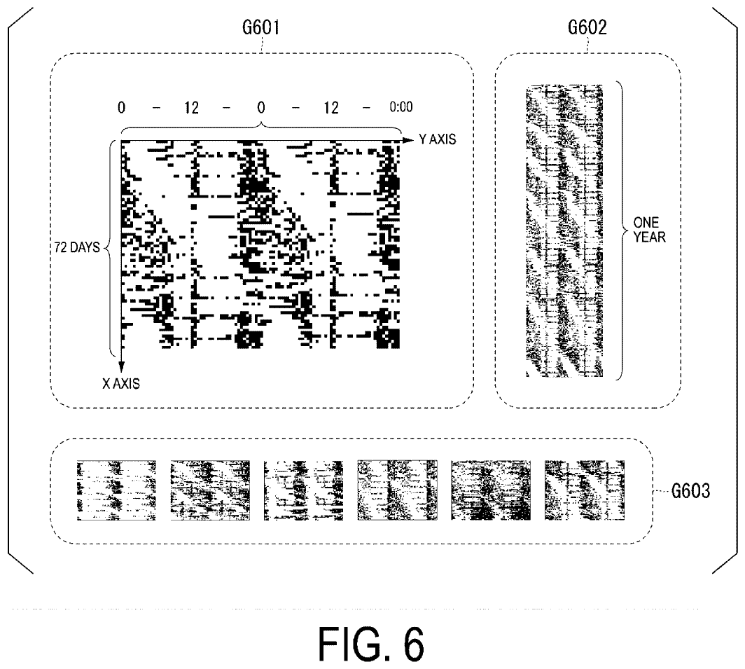

FIG. 6 illustrates an image G601, an image G602, and an image G603. The image G601 is the operation history information of the first user illustrating an example where the circadian rhythm indicates mixed disorder (24-hour sleep-wake+non-24-hour sleep-wake rhythm disorder). The image G603 is another example of the image G601. The image G602 illustrates an example of operation history information for one year for checking a difference from other examples of mixed disorder.

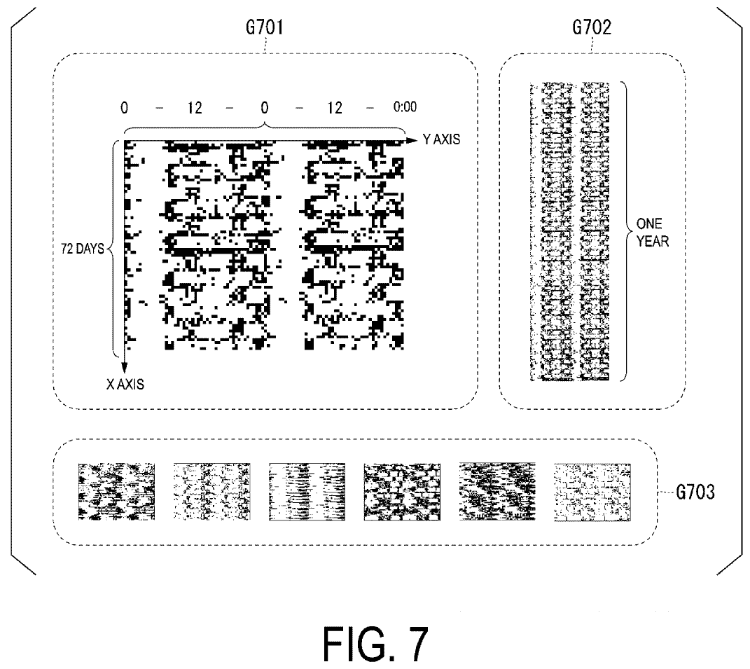

FIG. 7 illustrates an image G701, an image G702, and an image G703. The image G701 is the operation history information of the first user, illustrating an example in which the circadian rhythm corresponds to shift work. The image G703 is another example of the image G701. The image G702 illustrates an example of operation history information for one year for checking a difference from other examples of shift work.

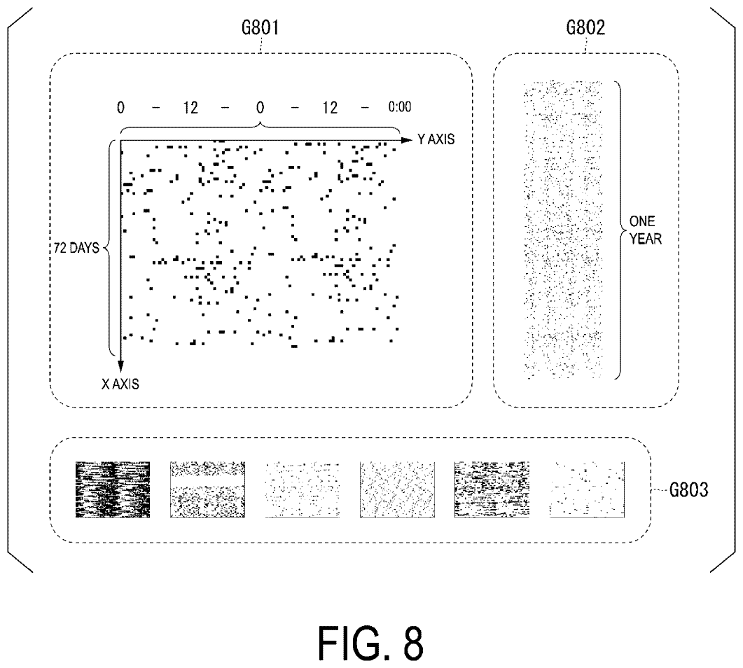

FIG. 8 illustrates an image G801, an image G802, and an image G803. The image G801 is the operation history information of the first user, illustrating an example in which the circadian rhythm corresponds to irregular sleep. The image G803 is another example of the image G801. The image G802 illustrates an example of operation history information for one year for checking a difference from other examples of irregular sleep.

Regarding each of the image G301, the image G401, the image G501, the image G601, the image G701, and the image G801, the x axis represents each date from the first day to the 72nd day, and the y axis represents 48 hours from 0:00 on each date indicated by the x axis to immediately before 0:00 two days later, between one end and the other end of the y axis. A point is plotted when an operation is performed within a 30 minute period. That is, if there is at least one operation within one 30 minute period, the plotting is done in response to the state regarded as “operated”, and if there is no operation within the period, the plotting is not done in response to the state regarded as “no operation”. The plotting may be done using something other than points, such as symbols or marks.

Therefore, each of the image G301, the image G401, the image G501, the image G601, the image G701, and the image G801 is an example of n=2 of the image representation condition. In the examples of the image G301, the image G401, the image G501, the image G601, the image G701, and the image G801, the left end of the y axis indicates 0:00 on each date indicated by the x axis, and the right end of the y axis indicates immediately before 0:00 two days after on each date indicated by the x axis.

The period of 72 days is the number of days considered necessary for preventing misrecognition as a wrong type of circadian rhythm. The number of days less than 72 days involves a higher risk of misrecognition as wrong type of circadian rhythm. Still, data is not required to be in all of the 72 days, and for example, even data lacks for several days due to trip, the machine learning processing and the classification processing can be executed. In order to appropriately execute the machine learning processing and the classification processing, it is desirable that two or more pieces of operation history information are present for one day.

Each of the image G301, the image G401, the image G501, the image G601, the image G701, and the image G801 is an image indicating that an operation has been performed on a predetermined operation target at the date and time when a point is plotted. In the example of these images, black dots are plotted, but the color of the dots may be any color other than the ground color. Therefore, each of the image G301, the image G401, the image G501, the image G601, the image G701, and the image G801 is information indicating an operation history of 72 days+1 day. As described above, the information indicating that an operation is performed is also information indicating that the circadian rhythm classification target is awake. Therefore, each of the image G301, the image G401, the image G501, the image G601, the image G701, and the image G801 indicating the operation history over a plurality of dates is also information indicating the sleep-wake rhythm of the circadian rhythm classification target.

The image G301 is the operation history information of the first user, illustrating the circadian rhythm indicating 24-hour sleep-wake for example. The 24-hour sleep-wake is a case where both the sleeping time and the wake-up time are in substantially constant time zones. In most cases, the 24-hour sleep-wake is a normal circadian rhythm. However, a late wake-up time (for example, around 12:00 PM) indicates a possibility of delayed sleep-wake phase disorder. In contrast, an early wake-up time (for example, around 2:00 AM) indicates a possibility of advanced sleep-wake phase disorder.

For example, the image G401 is an operation history information of the first user and indicates a possibility of circadian rhythm sleep-wake disorder with the circadian rhythm indicating the non-24-hour sleep-wake rhythm disorder (Straight). The non-24-hour sleep-wake rhythm disorder (Straight) is a circadian sleep-wake rhythm disorder in which the sleep-wake rhythm recedes for about one hour every day. The description “Straight” means that the receding interval is constant and appears to be linear in the image.

For example, the image G501 is the operation history information of the first user, and indicates a possibility of circadian sleep-wake rhythm disorder with the circadian rhythm indicating the non-24-hour sleep-wake rhythm disorder (Jump). The non-24-hour sleep-wake rhythm disorder (Jump) is a circadian sleep-wake rhythm disorder in which the sleep-wake rhythm recedes for about one hour every day. The description “Jump” means that the interval of recession is inconsistent and appears to be curved in an image.

For example, the image G601 is the operation history information of the first user, and indicates a possibility of a circadian sleep-wake rhythm disorder with the circadian rhythm indicating the mixed disorder (24-hour sleep-wake+non-24-hour sleep-wake rhythm disorder). The mixed disorder (24-hour sleep-wake+non-24-hour sleep-wake rhythm disorder) is a circadian sleep-wake rhythm disorder in which circadian rhythms indicating 24-hour sleep-wake and indicating non-24-hour sleep-wake rhythm disorder are mixed.

For example, the image G701 is operation history information of the first user, with the circadian rhythm indicating shift work. The shift work mentioned here is a case where a person is engaged in a shift work with a certain schedule, such as day shift, quasi night shift, or night shift. Shift work is a circadian rhythm artificially scheduled, but when the shift worker suffers sleeplessness or excessive sleepiness, there is a possibility of shift work disorder which is one of the circadian sleep-wake rhythm disorders.

For example, the image G801 is the operation history information of the first user with the circadian rhythm indicating a possibility of irregular sleep. The irregular sleep is a case where the sleeping time and the wake up time are inconsistent almost every day or the sleeping time and the wake up time are hardly recognized. The classification as irregular sleep and association with sleeplessness or excessive sleepiness indicates a possibility of irregular sleep-wake rhythm disorder which is one of the circadian rhythm sleep-wake disorders.

### <Diagram Illustrating Example of Hardware Configuration of Sleep Evaluation Device 1>

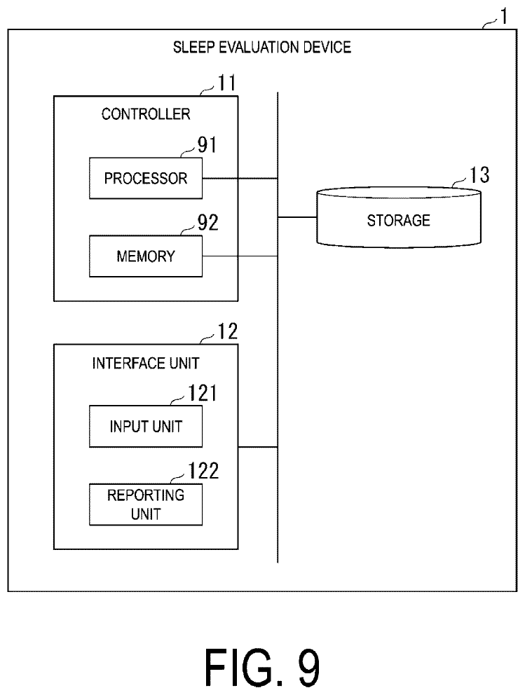

FIG. 9 is a diagram illustrating an example of a hardware configuration of the sleep evaluation device 1 according to the first embodiment. The sleep evaluation device 1 includes the controller 11 and executes a program. The sleep evaluation device 1 functions as a device including the controller 11, the interface unit 12, and a storage 13 by executing a program.

More specifically, the processor 91 reads a program stored in the storage 13 and stores the read program in the memory 92. When the processor 91 executes the program stored in the memory 92, the sleep evaluation device 1 functions as a device including the controller 11, the interface unit 12, and the storage 13.

The controller 11 controls operations of various functional units of the sleep evaluation device 1. The controller 11 executes, for example, the input acquisition processing, the machine learning processing, the classification processing, and the report control processing described above. The controller 11 records, for example, various types of information generated by execution of various types of processing in the storage 13. The controller 11 acquires, for example, information stored in the storage 13. The acquisition of the information recorded in the storage 13 is processing of reading the information stored in the storage 13 from the storage 13.

The interface unit 12 includes an input unit 121 and a reporter 122. The input unit 121 receives, for example, at least operation history information from an external device. In other words, the input unit 121 receives input of at least the operation history information from an external device. Therefore, the input unit 121 is a communication interface that is connected to at least the external device that is a transmission source of the operation history information, to be capable of performing wired or wireless communications. For example, the external device may be any device that is a transmission source of the operation history information, and may be the operation target itself, a predetermined terminal such as a smartphone, a personal computer, or smart glasses, a home appliance such as a refrigerator, a microwave oven, an air conditioner, a TV, or an electric fan, a remote controller, or a switch for turning ON or OFF the electricity in a room. Such an external device may be, for example, a server that stores an execution history of a program such as an interactive application or an electronic bulletin board as the operation target.

The input unit 121 may also be communicably connected to, for example, a device that transmits the circadian rhythm pattern labeled to the operation history information. In such a case, the input unit 121 acquires the operation history information and the circadian rhythm pattern labeled thereto, through communication with an external device that transmits the operation history information and the circadian rhythm pattern labeled thereto. A device that transmits such data may be, for example, a server or a terminal such as a personal computer. The training data means data in which the operation history information and the circadian rhythm pattern labeled to the operation history information are paired.

The input unit 121 may be a communication interface communicably connected to the transmission source device. In such a case, the input unit 121 acquires a pair of the operation history information and the circadian rhythm via wired or wireless communication with the transmission source device. The transmission source device may be, for example, a server or a terminal such as a personal computer operated by the creator of the training data.

When the input unit 121 acquires the operation history information with the circadian rhythm pattern labeled, the circadian rhythm classification model is trained using the operation history information and the circadian rhythm labeled to the operation history information. When the input unit 121 acquires operation history information without the circadian rhythm labeled, the classification processing is performed instead of the training.

The reporter 122 (an example of a reporting unit) is configured to include a display device such as a cathode ray tube (CRT) display, a liquid crystal display, or an organic electro-luminescence (EL) display. The reporter 122 may be configured to include a sound output device such as a speaker instead of such a display device, or may be configured to include the display device and the sound output device for example.

For example, the reporter 122 may output information input to the input unit 121 using an image or a sound. For example, the reporter 122 may output the result of the processing by the controller 11 using an image or a sound. The reporter 122 is an example of a reporter that reports the circadian rhythm classified by the classification processing under the control of the controller 11 to the second user.

The storage 13 is configured using a computer-readable storage medium device (non-transitory computer-readable recording medium) such as a magnetic hard disk device or a semiconductor storage device. The storage 13 stores various types of information related to the sleep evaluation device 1. The storage 13 stores, for example, information input via the interface unit 12. The storage 13 stores, for example, various types of information generated by the operation of the controller 11. Note that the storage 13 does not necessarily need to be provided in the sleep evaluation device 1, and may be present on a cloud, for example, as long as the controller 11 can communicate with the storage 13.

### <Diagram Illustrating Example of Configuration of Controller 11 of Sleep Evaluation Device 1>

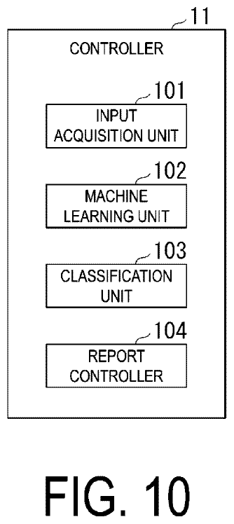

FIG. 10 is a diagram illustrating an example of a configuration of the controller 11 of the sleep evaluation device 1 according to the first embodiment. The controller 11 includes an input acquisition unit 101, the machine learning unit 102, the classification unit 103, and a report controller 104. The input acquisition unit 101 executes the input acquisition processing. The machine learning unit 102 executes the machine learning processing. The classification unit 103 executes the classification processing. The report controller 104 executes the report control processing.

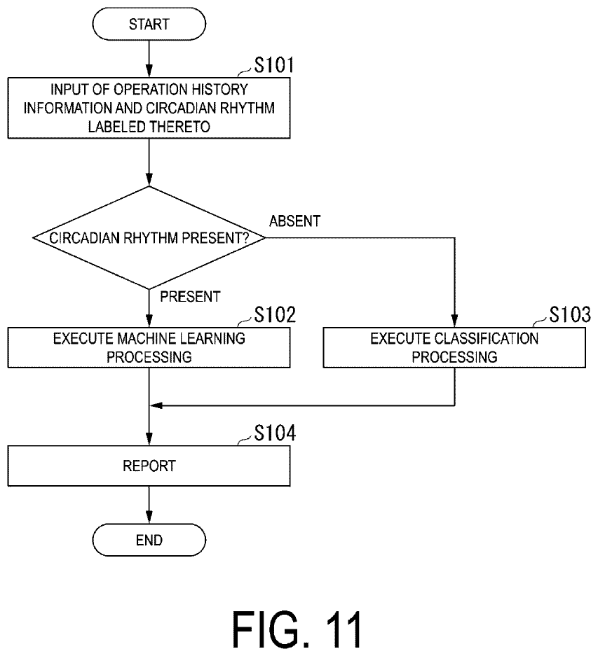

FIG. 11 is a flowchart illustrating an example of a flow of processing executed by the sleep evaluation device 1 according to the first embodiment. The operation history information and the circadian rhythm labeled thereto are input to the input unit 121 (step S101). Here, as the operation history information, the operation history information of the first user and the operation history information of the second user are input. Note that step S101 is an example of an input step of receiving the operation history information, which is information indicating an operation history, from an external device.

When the training data including the operation history information and the circadian rhythm pattern labeled to the operation history information is input to the input unit 121, the machine learning unit 102 executes the machine learning processing based on the training data including the input operation history information of the first user and the circadian rhythm pattern labeled to the operation history information (step S102). The circadian rhythm pattern paired with the operation history information of the first user does not necessarily need to be input from the input unit 121 in step S101. For example, the circadian rhythm pattern stored in advance in a predetermined storage device such as the storage 13 may be used, or the circadian rhythm pattern may be acquired from a predetermined external device.

Thus, the machine learning unit 102 performs supervised learning for the circadian rhythm classification thereby a model is obtained to classify a circadian rhythm based on the training data including the input operation history information of the first user and the circadian rhythm pattern labeled thereto. The step S102 is an example of a machine learning step of performing supervised learning for the circadian rhythm classification thereby a model is obtained to classify a circadian rhythm based on training data including the input operation history information of the first user and the circadian rhythm pattern labeled thereto.

If there is operation history information in the input unit 121 without the circadian rhythm pattern labeled thereto, the classification unit 103 executes the classification processing based on the operation history information of the second user (step S103). Thus, the classification unit 103 classifies the circadian rhythm of a second user based on the operation history information of the second user input and the circadian rhythm classification model. Therefore, step S103 is an example of a classification step of classifying the circadian rhythm of the second user based on the operation history information of the second user input and the circadian rhythm classification model.

Next, the reporter 122 reports the circadian rhythms classified by the classification unit 103 to the second user (step S104). The step S104 is an example of a reporting step of reporting the circadian rhythms classified in the classifying step to the second user.

The processing in one of steps S102 and S103 is executed depending on the presence or absence of the circadian rhythm patterns labeled to the operation history information. Further, step S104 may be executed after execution of step S103.

The sleep evaluation device 1 configured as described above executes the classification processing. Therefore, as described in the above, the sleep evaluation device 1 can provide an opportunity to visit a medical institution at an early stage by pointing out a symptom suspected to be circadian sleep-wake rhythm disorder as described in <Example of Effect of Controller 11> above.

### Second Embodiment

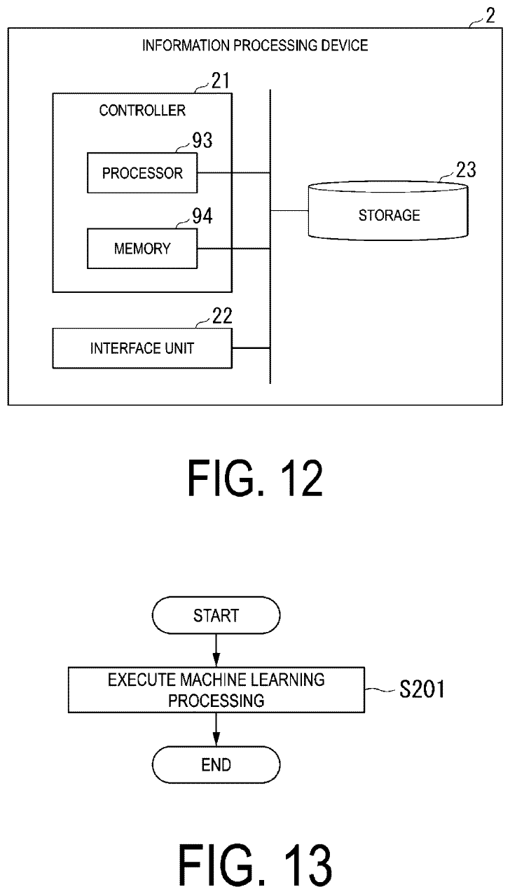

FIG. 12 is a diagram illustrating an example of a hardware configuration of an information processing device 2 (an example of a learning device) according to a second embodiment. The information processing device 2 includes a controller 21 including a processor 93 such as a CPU, a GPU, or an NPU and a memory 94 which are connected to each other by a bus, and executes a program. The information processing device 2 functions as a device including the controller 21, an interface unit 22, and a storage 23 by executing a program.

More specifically, the processor 93 reads a program stored in the storage 23 and stores the read program in the memory 94. When the processor 93 executes the program stored in the memory 94, the information processing device 2 functions as a device including the controller 21, the interface unit 22, and the storage 23.

The controller 21 controls operations of various functional units of the information processing device 2. The controller 21 executes, for example, machine learning processing. The definition of the machine learning processing is the same as the definition of the machine learning processing in the first embodiment. The definition of the operation history information is the same as the definition in the first embodiment. The definition of the circadian rhythm is also the same as the definition in the first embodiment. Therefore, the definition of the training data used for training the circadian rhythm classification model is also the same as the definition in the first embodiment. Further, as in the first embodiment, when the operation history information is represented by an image, the image may be an image satisfying the image representation condition described in the first embodiment.

The controller 21 acquires, for example, information stored in the storage 23. The acquisition of the information recorded in the storage 23 is processing of reading the information stored in the storage 23 from the storage 23.

The interface unit 22 includes a communication interface for connecting the information processing device 2 to an external device. The interface unit 22 communicates with an external device wirelessly or using a wire.

The external device is, for example, a device that is a transmission source of a signal or information used for the training. The interface unit 22 acquires a signal or information used for the training through communication with a device that is a transmission source of the signal or information used for the training. The signal or information used for the training is, for example, training data. In other words, the signal or information used for the training is, for example, a set of operation history information and a circadian rhythm pattern labeled thereto.

The external device may be a classification device, for example. The classification device is a device that classifies a circadian rhythm (that is, executes the classification processing) using the trained circadian rhythm classification model obtained by the information processing device 2. In such a case, the classification device can execute the trained circadian rhythm classification model obtained by the information processing device 2 through communication via the interface unit 22. The classification device may output a result of the classification processing to a predetermined output destination.

The interface unit 22 may include an input device such as a mouse, a keyboard, or a touch panel. The interface unit 22 may be configured as an interface that connects these input devices to the information processing device 2. Thus, the input device of the interface unit 22 receives input of various types of information to the information processing device 2 wirelessly or using a wire. The signal or the information does not necessarily need to be input to the communication interface of the interface unit 22 and may be input to the input device of the interface unit 22.

The interface unit 22 outputs, for example, various types of information. The interface unit 22 is configured to include a display device such as a CRT display, a liquid crystal display, or an organic EL display, and a speaker for example. The interface unit 22 may be configured as an interface that connects the display device or the speaker to the information processing device 2. Therefore, the interface unit 22 may output, for example, information input to the input device of the interface unit 22 using an image or a sound.

The storage 23 is configured using a computer-readable storage medium device such as a magnetic hard disk device or a semiconductor storage device. The storage 23 stores various types of information related to the information processing device 2. The storage 23 stores, for example, information input via the interface unit 22. The storage 23 stores, for example, various types of information generated by the operation of the controller 21. Note that the storage 23 does not necessarily need to be provided in the information processing device 2, and may be present on, for example, a cloud as long as the controller 21 can communicate with the storage 23.

FIG. 13 is a flowchart illustrating an example of a flow of processing executed by the information processing device 2 according to the second embodiment. The controller 21 executes the machine learning processing (step S201).

The controller 21 may execute the classification processing. The classification processing executed by the controller 21 is the same as the classification processing in the first embodiment. In such a case, the controller 21 may control the operation of a predetermined output destination such as the interface unit 22 to output the result of the classification processing for example.

Therefore, the interface unit 22 of the information processing device 2 may further include, for example, the reporter 122. In such a case, the controller 21 may control the operation of the reporter 122 to report the result of the classification processing.

### <Example of Effect of Information Processing Device 2>

The information processing device 2 includes the controller 21 that executes machine learning processing. The circadian rhythm classification model, which is a model is obtained by the machine learning processing, is a model for classifying a circadian rhythm of a circadian rhythm classification target (that is, a user). The machine learning improves the accuracy of circadian rhythm classification. If the type of a circadian rhythm can be recognized with higher accuracy, a possibility that the user reviews his/her lifestyle habits or visits a medical institution increases. As a result, the possibility of improvement of circadian sleep-wake rhythm disorder increases. Therefore, the information processing device 2 can increase the possibility of improvement for circadian sleep-wake rhythm disorder. Thus, the information processing device 2 can provide a technique for increasing the possibility of improvement for circadian sleep-wake rhythm disorders.

The information processing device 2 according to the modification configured as described above includes the controller 21 that executes the machine learning processing. By using the trained model obtained by executing the machine learning processing, it is possible to point out a symptom suspected to be circadian sleep-wake rhythm disorder. By pointing out a symptom suspected to be a circadian sleep-wake rhythm disorder using a trained circadian rhythm classification model, an opportunity to visit a medical institution can be provided. In addition, the labor and time required for machine learning can be saved.

In addition, since the information processing device 2 according to the modification configured as described above includes the controller 21 that executes the machine learning processing, it is possible to provide a technique for increasing the possibility of improvement for circadian sleep-wake rhythm disorder, as described in <Example of Effect Provided by Information Processing Device 2>.

The sleep evaluation device 1 may be implemented by using a plurality of information processing devices communicably connected via a network. In this case, each processing executed by the sleep evaluation device 1 may be executed by a plurality of information processing devices in a distributed manner.

Note that the information processing device 2 may be implemented by using a plurality of information processing devices communicably connected via a network. In this case, each processing executed by the information processing device 2 may be executed by a plurality of information processing devices in a distributed manner.

All or a part of the functions of the sleep evaluation device 1 and the information processing device 2 may be implemented using hardware such as an application specific integrated circuit (ASIC), a programmable logic device (PLD), or a field programmable gate array (FPGA). The program may be recorded in a computer readable medium. The computer-readable recording medium includes a portable medium such as a flexible disk, a magneto-optical disk, a ROM, and a CD-ROM, and a storage device such as a hard disk incorporated in the computer system for example. The program may be transmitted via an electric communication line.

The first embodiment and the second embodiment of the invention are described above in detail with reference to the drawings. However, specific configurations are not limited to the first embodiment and the second embodiment and also include design or the like without departing from the gist of the invention.

### REFERENCE SIGNS LIST

1 . . . sleep evaluation device, 11 . . . controller, 12 . . . interface unit, 13 . . . storage, 121 . . . input unit, 122 . . . reporter, 101 . . . input acquisition unit, 102 . . . machine learning unit, 103 . . . classification unit, 104 . . . report controller, 2 . . . information processing device, 21 . . . controller, 22 . . . interface unit, 23 . . . storage, 91 . . . processor, 92 . . . memory, 93 . . . processor, 94 . . . memory

1. A sleep evaluation device, comprising:
a processor; and
a storage medium having computer program instructions stored thereon, wherein the computer program instruction, when executed by the processor, perform processing of:
receiving operation history information that is information indicating an operation history and a circadian rhythm pattern labeled to the operation history information from an external device;
performing supervised learning for a circadian rhythm classification thereby a model is obtained to classify a circadian rhythm based on training data including the operation history information of a first user and the circadian rhythm pattern labeled to the operation history information received;
classifying a circadian rhythm of a second user based on the operation history information of the second user received and the circadian rhythm classification model; and
reporting to the second user, the circadian rhythm classified by the classification.

2. The sleep evaluation device according to claim 1, wherein when the operation history information is data indicating date and time, the processor plots a point indicating the date and time when an operation is performed, on a white background image formed of pixels in a predetermined range, and uses the white background image after the plotting as the operation history information.

3. The sleep evaluation device according to claim 2, wherein
one of a vertical axis and a horizontal axis in a coordinate of the white background image indicates a date and another one of the vertical axis and the horizontal axis indicates time, and
the range of time axis is n times longer than 24 hours, n being an integer that is one or more.

4. A program causing a computer, as the sleep evaluation device according to claim 1, to execute functions of:
performing supervised learning for a circadian rhythm classification thereby a model is obtained to classify a circadian rhythm based on training data including the operation history information of a first user and the circadian rhythm pattern labeled to the operation history information received, and
classifying a circadian rhythm of a second user based on the operation history information of the second user received and the circadian rhythm classification model.

5. A sleep evaluation method, comprising:
receiving operation history information that is time-series data indicating an operation history and a circadian rhythm pattern labeled to the operation history information from an external device;
performing supervised learning for a circadian rhythm classification thereby a model is obtained to classify a circadian rhythm based on training data including the operation history information of a first user and the circadian rhythm pattern labeled to the operation history information received;
classifying a circadian rhythm of a second user based on the operation history information of the second user received and the circadian rhythm classification model; and
reporting to the second user, the circadian rhythm classified by the through the classifying.

6. A learning device, comprising:
a processor; and
a storage medium having computer program instructions stored thereon, wherein the computer program instruction, when executed by the processor, perform processing of:
performing learning for a circadian rhythm classification thereby a model is obtained to classify a circadian rhythm for a user that has operated the device, based on training data including operation history information that is time-series data indicating an operation history output from an external device and a circadian rhythm pattern labeled to the operation history information.
